(19) United States
(12) Patent Application Publication
(10) Pub. No.: US 2020/0231928 A1
(43) Pub. Date: Jul. 23, 2020

(54) REMEDIATION TECHNIQUES USING COMPOSITES HAVING NOVEL ORGANIC COMPONENTS

(71) Applicant: Marcos GONZALEZ, Davie, FL (US)

(72) Inventor: Marcos GONZALEZ, Davie, FL (US)

(21) Appl. No.: 16/252,686

(22) Filed: Jan. 20, 2019

(51) Int. Cl. C12N 1/12; C12N 1/20; C02F 3/32; B09C 1/10

(52) U.S. Cl. CPC C12N 1/12; B09C 1/105; C02F 3/325; C12N 1/20

(57) ABSTRACT

Various methods for remediating materials/mediums are disclosed. For example, a particular method may include growing an amount of cyanotic organisms and red algae, separating cell walls of the cyanotic organisms from internal portions of the cyanotic organisms to form a separated cyanotic extract, the separated cyanotic extract being the internal portions of the cyanotic organisms, separating cell walls of the red algae from internal portions of the red algae to form a separated red algae extract, the separated red algae extract being the internal portions of the red algae, processing the separated cyanotic extract and processing the separated red algae extract so as to create a hybrid algae product, and performing a pollution remediation operation using the hybrid algae product.

This application takes priority from, and incorporates the entire content of, U.S. Provisional Application No. 61/700,280 entitled “COMPOSITES HAVING NOVEL ORGANIC COMPONENTS AND METHODS OF MANUFACTURE” filed on Sep. 12, 2012, by inventor Marcos Gonzalez. This application takes priority from and incorporates the entire content of PCT Application No. PCT/US13/34404 entitled “COMPOSITES HAVING NOVEL ORGANIC COMPONENTS AND METHODS OF MANUFACTURE” filed on Mar. 28, 2013, by inventor Marcos Gonzalez. This application also takes priority from, and incorporates the entire content of, U.S. Non-Provisional application Ser. No. 14/428,369 entitled “COATING COMPOSITES HAVING NOVEL ORGANIC COMPONENTS AND METHODS OF MANUFACTURE” filed on Mar. 15, 2015, by inventor Marcos Gonzalez. This application also takes priority from, and incorporates the entire content of, U.S. Provisional Application No. 62/717,810 entitled “COATING COMPOSITES HAVING NOVEL ORGANIC COMPONENTS AND METHODS OF MANUFACTURE” (Docket No. 100100-005P) filed on Aug. 11, 2018, by inventor Marcos Gonzalez.

### BACKGROUND

### I. Field

This disclosure relates to methods and systems for performing pollution remediation.

### II. Background

Pollution remediation is an underdeveloped technology—mostly due to the expense required to clean up even minor amounts of pollution. Further, in certain circumstances no effective solutions have been found related to the remediation of farm soils, fracking water, etc.

### BRIEF DESCRIPTION OF THE DRAWINGS

The features and nature of the present disclosure will become more apparent from the detailed description set forth below when taken in conjunction with the accompanying drawings in which reference characters identify corresponding items.

FIG. 1 is an example bioreactor usable to grow green and/or cyanotic organisms and harvest organic extracts.

FIG. 2 depicts example equipment used to process the organic extracts of FIG. 1.

FIG. 3 depicts example equipment used to further process the organic extracts of FIGS. 1-2 so as to create a dry-storage form of the organic extract.

FIG. 3B depicts a substrate particle covered with a balanced algae extract coating.

FIG. 4 is a flowchart outlining an exemplary operation for growing cyanotic organisms and harvesting organic extracts.

FIG. 5 is a flowchart outlining an exemplary operation for remediating a polluted medium.

FIG. 6 is a graph of the PH of a medium subjected to the disclosed remediation methods over time.

### DETAILED DESCRIPTION

The disclosed methods and systems below may be described generally, as well as in terms of specific examples and/or specific examples. For instances where references are made to detailed examples and/or examples, it should be appreciated that any of the underlying principals described are not to be limited to a single example but may be expanded for use with any of the other methods and systems described herein as will be understood by one of ordinary skill in the art unless otherwise stated specifically.

The following definitions apply for this disclosure:

The term “aggregate” refers to a component of a composite material used to resist compressive or tensile stresses.

The term “structural composite” refers to a concrete, a geopolymer, an elasto-polymer or a cryptobiotic material.

The term “polymer” refers to a class of materials composed of repeating structural units. These structural units are typically connected by covalent chemical bonds. Although the term “polymer” is sometimes taken to refer only to plastics, the term encompasses a large class of compounds with a wide variety of properties.

The term “biomineralization” refers to a process by which certain living organisms take various compounds from their environment to produce specialized minerals—often to harden or stiffen various tissues. Past man-made attempts to produce similar minerals that various organisms produce easily have largely been a failure as they are energy inefficient, and require stringent conditions, such as high temperature, pressure or extreme pH levels.

The class of materials disclosed below are referred to as “Elasto-Polymers,” (“EPs”) or “Geo-Elasto-polymers,” (“GEPs”) and are meant to refer to a class of materials that can take anything from a slightly elastic to rock-like form, and which is based in part on what is believed to be a synthetic biomineralization using organic molecules and/or induced covalent bonding cased by organic substances.

The organic components in the materials described below are taken from any of a variety of green or blue-green alga species, such as Synechocystis. It is to be appreciated that the terms “cyanobacteria” and “blue-green algae” are often, but not always, used synonymously in science and industry. The American Heritage dictionary defines cyanobacteria as follows: “Cyanobacteria Cy⋅a⋅no⋅bac⋅te⋅ri⋅a (sī [symbol] -nō-bāk-tēr′ē- [symbol] ) n. A group of Procaryotae consisting of unicellular or filamentous gram-negative microorganisms that are either nonmotile or possess a gliding motility, may reproduce by binary fission, and photosynthetically produce oxygen; some species capable of fixing nitrogen. Members of this phylum were formerly called blue-green algae.” The American Heritage® Stedman's Medical Dictionary (2002)

However, there may be modern distinctions between cyanobacteria and eukaryotic organisms (algae) based on anatomical differences not relevant to this disclosure. It is to be appreciated in light of the following disclosure that the two terms are meant to be equivalents given their traditional, rather than recently emerged, definitions, unless otherwise stated or claimed.

However, for the purposes of distinguishing the two types of organisms, when necessary, to differentiate different attributes of the two classes of organisms (cyanobacteria and eukaryotic organisms), and for the sake of claiming, the terms will be considered as different organisms while the generic term “cyanotic organism” shall be used to refer to both organisms, as well as later equivalent organisms that may later be discovered, synthetically developed (e.g., genetically engineered or selectively bred), or later recognized.

Another distinction to be made is that of single-cell algae and seaweed. “Seaweed” is a loose colloquial term encompassing macroscopic, multicellular, benthic marine algae. The term includes some members of the red, brown and green algae. For the purpose of this document, cyanotic organisms are considered to be distinct from seaweed.

A particular ingredient useful in the production of concrete and elastopolymers is known as a “plasticizer.” Plasticizers are water reducers or dispersants that can be added to concrete or other composite mixtures to improve workability. Generally, the strength of a concrete is inversely proportional to the amount of water added or water-cement (w/c) ratio. In order to produce a stronger concrete/composite, less water is added. Too little water, however, “starves” the mix, which makes the concrete/composite mixture very unworkable and difficult to mix, necessitating the use of plasticizers.

This disclosure describes a novel process and mixtures capable of producing structural composites suitable as a building material. The process and resultant materials can produce structures that are both low-cost and durable compared to structures made of conventional building materials, such as Portland cement or geopolymers.

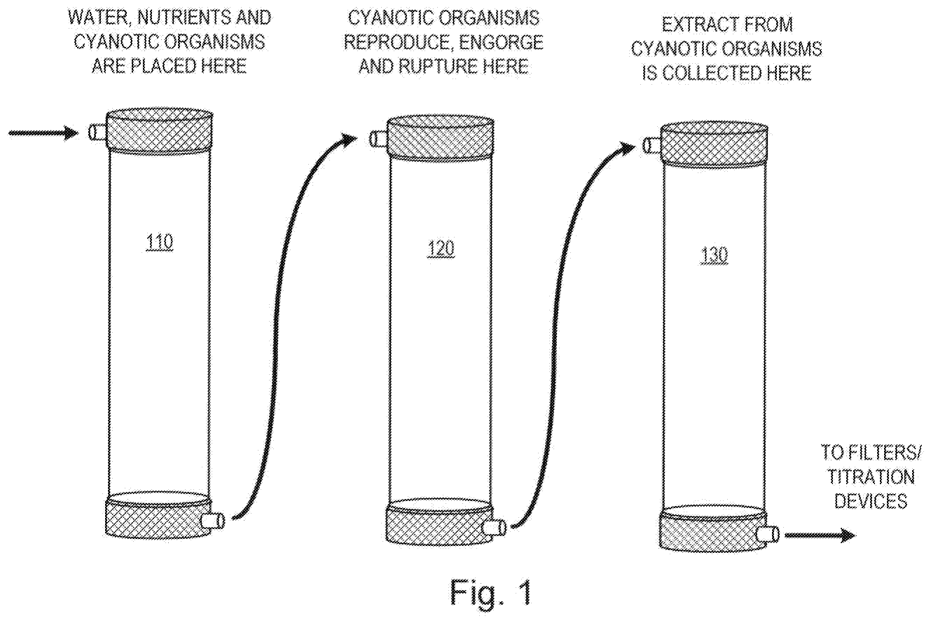

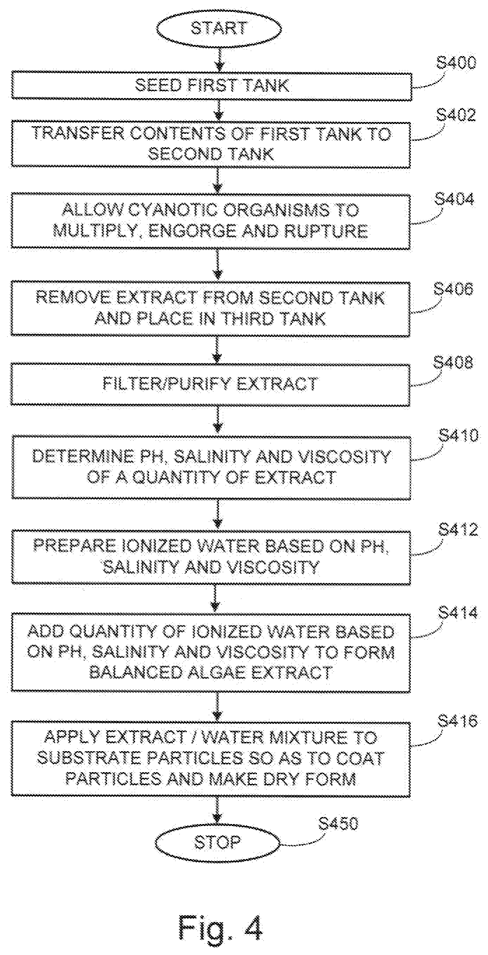

FIG. 1 is an example bioreactor 100 usable to grow green algae and/or cyanotic organisms and harvest organic extracts. The example bioreactor 100 includes three tanks including a first tank 110, a second tank 120 and a third tank 130. Usage of the tanks 110, 120 and 130 will be discussed below with reference to FIG. 4 (steps S400-S406), which outlines a method for growing green and/or cyanotic organisms, and harvesting a resultant algae extract.

The process of FIG. 4 starts in step S400 where one or more targeted species of green or cyanotic organisms is cultured. In the non-limiting example of this disclosure, the cyanotic organisms used may include Synechocystis (PCC 6803) and/or any number of species of the collenia genus noting that there may be a wide range of suitable microbes, including microbes more recently distinguished as algae. Because these organisms can reproduce asexually, they can be cultured in bioreactors by a photosynthesis process. In the present example, the first tank 110 is used to culture the selected cyanotic organisms by placing them in a solution of water and nutrients, such as any number of proteins (e.g., laminin, myosin, collagen, actin and/or keratin), selenium, triphosphates, magnesium sterate, salt, magnesium oxide, methosol, barium, carbon dioxide, and so on. The particular mixture of nutrients can vary according to a large range of criteria, such as the type of organism(s) used and varying environmental conditions.

At step S402, the medium and cyanotic organisms are transferred from the first tank to a second tank (such as tank 120 of FIG. 1) where in step S404 the cyanotic organisms are allowed to multiply, engorge and rupture as a result of over-engorgement within limited space. It is to be appreciated, however, that the cell walls of the organisms may be broken by any number of known or later-developed processes, such as mechanically slicing cell walls or rupturing cell walls by some other mechanism, such as a high-speed blending process, application of ultra-sonic energy, chemical treatment, and so on.

Next, in step S406, the algae extract, which contains various yet-unidentified and identified enzymes and proteins, are removed from the second tank and transferred to a third tank (e.g., tank 130 of FIG. 1), where the algae extract is stored.

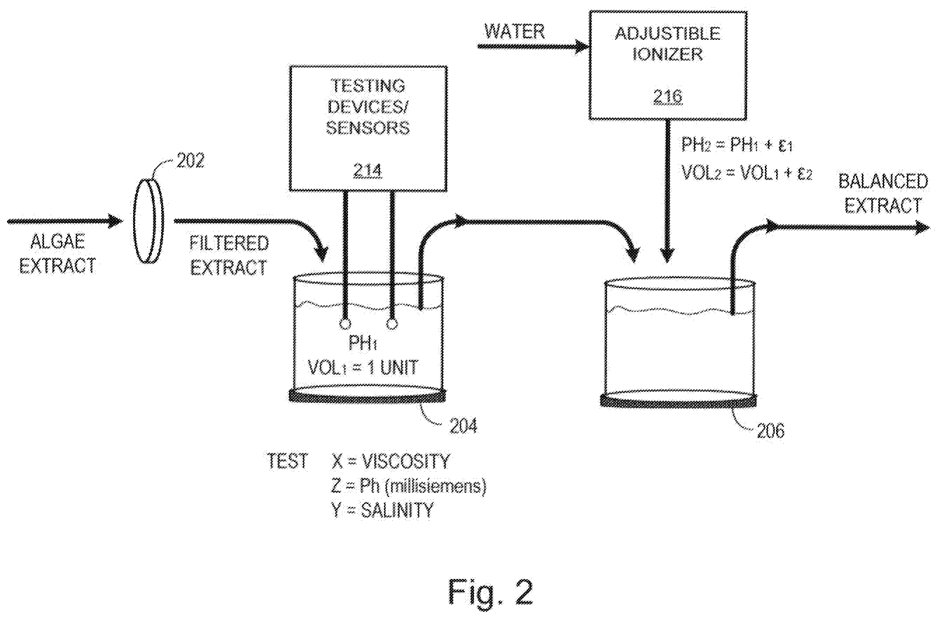

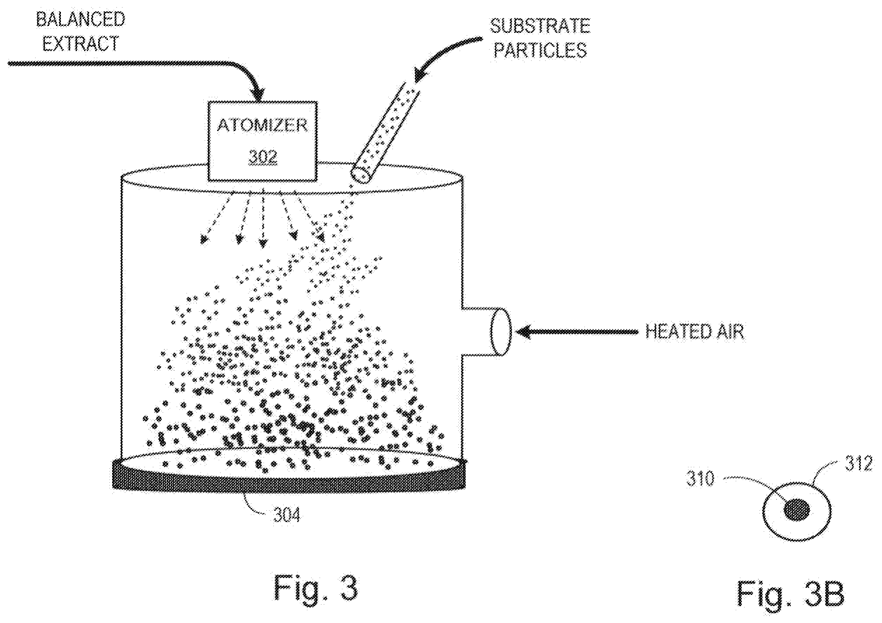

FIGS. 2-3 depict example equipment used to process the organic extracts of FIG. 1 and steps S400-S406. Such example equipment includes a filter 202, a first storage unit 204, testing equipment 214, a second storage unit 206, an adjustable ionizer 216, coating chamber 304 and atomizer 302. Usage of devices 202-304 will be discussed below with reference to FIG. 4 (steps S408-S450), which outlines a method for processing the algae extract of steps S400-S406.

In step S408, the algae extract is further processed/purified by various filtering procedures intended to remove algae cell walls and other contaminants. Such filtering can be performed with, for example, the filter 202 of FIG. 2. Filter 202 may take the form of a membrane filter, other forms of filters, a centrifuge or any other known or later-developed device capable of separating the interior of algae cells from respective cell walls and other contaminants. For the purpose of this disclosure, the filtered portion of the extract may be referred to as a “filtered extract” or “purified extract.” It is to be appreciated that the purified extract may now be optionally combined with any number of proteins, e.g., laminin, myosin, collagen, actin and/or keratin.

Next, in step S410, the filtered/purified extract, which can take the form of a generally clear viscous syrup, is tested to determine viscosity, pH and salinity, which can vary from batch to batch as a function of different algae types, different nutrients added and different environmental conditions. Testing can be performed by the testing devices/sensors 214 of FIG. 2, which can include, for example, a viscometer or rheometer, a digital pH meter or pH test strips, and any number of commercially available salinity testing devices.

Next, in step S412, a quantity of ionized water is prepared based on the viscosity, pH and/or salinity. Such ionized water can be produced using, for example, the adjustable ionizer 216 of FIG. 2. It is expected that the pH of the purified extract (defined as pH₁) will be positive/alkaline. Accordingly, the pH of water produced by the adjustable ionizer 216 (defined as pH₂) will also be alkaline and carry a substantial number of hydroxide (OH—) ions. Generally, pH₂=pH₁+ε₁, with s, being defined as a first error-adjustment factor that will vary as a function of the salinity and viscosity. Generally, ε₁ will increase with viscosity and increase with salinity, but the exact amount of adjustment/error may vary according to the algae type(s) and nutrients used. For the purpose of claim construction, the term “generally equal” as it pertains to relative volumes refers to an expected range about a median value that can be determined by one of ordinary skill in the art using a particular set of conditions. As variables, such as viscosity, can easily change depending on, for example, equipment used, environmental conditions (and ranges thereof), nutrient mix and cyanotic organism, the range of “generally equal” should be determined using an experimental baseline according to detailed algae production controls.

As with the pH, the quantity/volume of added ionized water can vary. Generally, the amount of added ionized water (VOL₂)=the volume of purified extract (VOL₁)+ε₂, with ε₂ being defined as a second error-adjustment factor that will vary as a function of the salinity and viscosity. Generally, ε₂ will increase with viscosity and increase with salinity, but the exact amount of adjustment/error again may vary according to the algae type(s) and nutrients used.

Then, in step S414, the quantity/volume (VOL₂) of ionized water is added to the quantity/volume (VOL₁) of purified extract. In this step, the ionized water, when properly prepared and mixed to the purified algae extract, causes the purified extract to be “structurally balanced” so as to stop the purified extract from reacting with its environment and become chemically static. That is, enzyme molecules normally in a transient/changing state are stabilized by the hydroxide ions by causing the enzyme molecules to become polarized so as to structurally repel other molecules in suspension. Thus, the algae extract has little reactivity or no chemical reactivity with the term “little reactivity” meaning that there is a low enough level of chemical degradation such that the balanced algae extract would be still commercially viable after at least several weeks of storage.

In step S416, the balanced extract can be applied to a carrier to make a “Dry Form Plasticizer” (“DFP”), which has a commercial advantage of being in an industry familiar form capable of being easily stored, re-hydrated and/or mixed with aggregates.

FIG. 3 depicts an example apparatus where substrate particles, such as round, smooth particles of silicon 310 (or another mineral) are covered with a balanced extract coating 312. See, FIG. 38. In operation, the balanced algae extract is passed through an atomizer 302 into tank 304. Substrate particles, such as round, smooth particles of silicon 310, are subjected to the balanced extract. By virtue of hot air injected into tank 304, a coating of increasing size will accumulate around the individual particles until the coated particles become large and heavy enough to fall to the bottom of tank 304, where they can layer be removed.

The nature of the DFP is two-fold. First, it provides an excellent non-toxic and natural plasticizer when making composite materials, such as concrete. Second, when made from a properly balanced algae extract, the DFP has an unexpected property of forcing covalent bonds among a large variety of substances, and can compel what appears to be a form of biomineralization of organic materials, such as sawdust and seed husks.

The balanced dry-form extract/plasticizer, when used as a plasticizer for a composite material, initially compels a hydrophilic reaction by absorbing water. Subsequently, the same extract causes a hydrophobic reaction of the composite material by expelling water as covalent bonds are formed by the resultant composite material. The second process occurs when added water disrupts the previously attained pH balance in step S414.

The steps above can produce polycarboxylate ethers (PCE) or just polycarboxylate (PC). PCE/PC represents a new generation of plasticizers that are not only chemically different from the older sulfonated melamine and naphthalene-based products, but their action mechanism is also different. That is, PCE/PC can act by causing particle dispersion of a composite by steric stabilization. In contrast, conventional plasticizers use an electrostatic repulsion effect to achieve particle dispersion.

The steric stabilization form of dispersion is more powerful in its effect and gives improved workability retention to the cementitious mix. Furthermore, the chemical structure of PCE allows for a greater degree of chemical modification than the older-generation products, offering a range of performance that can be tailored to meet specific needs.

### INDUSTRIAL APPLICATIONS

The extract (or any number of modified or purified extracts) may be added to an aggregate mixture for concrete or any number of concrete or elastopolymer formulations. With regard to elastopolymers, a theory arises that sepiolite (e.g., polygorskite with a biofilm blend of enzymes and genus species of cryptomatic soils and cyanobacteria cultures) can act as carrier minerals that break down as the alkalinity rises, thus delivering soluble and mobile SiO₂ and Al₂O₃ for pozzolanic reactions that form more polymeric minerals. In various examples, the reaction may be accelerated with reactive magnesium oxide, which can produce stronger bonds and control the PH during the reaction curve. Polycondensation of organic substances (natural polymers, proteins, enzymes and minerals produced organic and inorganically by chemical reactions that result in cross-linked through hydrogen bonding forming covalent bonds of aggregates and materials, which in turn may form cryptomatic composites.

The combination of the biofilm (enzymes) produced by the algae microbes with keratin and laminin can produce an effective bonding agent by geosynthesis to, in turn, create covalent bonds with a cross-linking matrix in combination with conventional aggregates when combined with aluminum silacate, selenium, humic acid, sodium metasilrate reactive magnesium oxide and fly ash.

### EXAMPLES

### Example 1

a combination of 10% DFP (by weight) can be added to 30% pozzolanic materials, 30% AlSi (fly ash) and 30% limestone power can be mixed, with some other aggregate(s), e.g., sand or stones, to create a concrete-like material with compression and tensile strength superior to Portland cement. The resultant material may be made with a near-zero co-efficient of expansion, impervious to water, highly resistant to corrosives, such as acid. For example, a cube of the resultant composite was subjected to a 30% HCL bath for 160 hours, but lost only 0.2% mass. In contrast, a similar-sized cube of Portland was dissolved to a mushy consistency. The resultant composite is estimated to be 20%-30% cheaper to manufacture than Portland, and has a negative carbon footprint.

### Example 2

a wood substitute may be made using a combination of 10% DFP (by weight), 20% pozzolanic materials, 50% fly ash and 20% silica. Aggregates, such as sawdust, seed husks or some other cellulose-bearing material, may be added. The resultant material may be worked much as wood can be worked, but will not burn even with the application of an oxy-acetylene torch. The resultant material has a mechanical flexibility similar to wood, but is not subject to rot.

### Example 3

a combination of 20% DFP (by weight) can be added to 50% AlSi, and 30% metallic grindings/powders, such as zinc, iron and copper. The resultant material may be made with a near-zero co-efficient of expansion, impervious to water, highly resistant to corrosives, such as acid.

### Example 4

an extract may be applied to parallel strands of hollow and porous basalt fibers, then allowed to cure to a rebar-like form. The rebar-equivalent is about ⅙^{th} the weight, cheaper to manufacture, impervious to corrosion and has a far superior tensile strength.

### Example 4

an extract may be applied to a flat weave of hollow and porous basalt fibers, then allowed to cure. The resultant product resembles fiberglass composites, but with better strength and insulative properties.

### Example 5

a composite similar to that of example 1 may be made with increased amounts of DFP. The resultant product resembles concrete, but has a capacity to better absorb vibrations, which can be valuable to produce foundations for generators or motors as it will extend the service life of internal moving parts, such as bearings.

The percentage of extract may vary, but it is envisioned that an effective amount of extract may, depending on variations of aggregates and chemistry, range from about 0.5% to 5%, e.g., 1%-3%. Adding greater amounts of the extract can cause the resultant material to take on more plastic-like qualities.

Distinct Algae-Based Products

For the purpose of this disclosure, there are at least three algae-based materials that may be produced according to the disclosed methods and systems which are herein defines as:

(1) C Product—being derived from any number of cyanotic organisms;

(2) G Product—being produced by any number of green algae species; and

(3) R Product—being produced by any number of red algae species and possibly some brown algae species.

Each of the C. G and R products may be produced using the methods and devices described above noting that, for the purposes of this disclosure, none of the C Product, the G Product or the R Product is produced from organisms classified as seaweed. Thus, the devices of FIGS. 1-3, and the method of FIG. 4 are to be considered just as applicable to processing green algae and red algae.

It is to be appreciated that various algae species will produce products having different properties and uses. As to properties, for example, the C Products will have a PH ranging from about 8.5 to 14.0; the G Products will have a PH ranging from about 5.5 to 8.5; and the R Products will have a PH ranging from about 2.5 to 5.5.

However, it may be important to provide a specific algae-based product having a specific PH, which may not be easily accomplished using any one species of algae.

To address this issue of creating an algae product having a specific PH, one approach is to mix processed C Product and/or G Product with Product in proportions that provide an appropriate PH.

However, in other examples, it may be useful to grow different algae species in a single medium. For example, it can be problematic as different algae types are often destructive/toxic to one another. For example, blue-green algae and red algae are generally incompatible with one another, and blue-green algae generally reproduces more rapidly so as to out-compete red algae.

However, to address this problem, it has been determined that various red algae species may be grown in a same medium as blue-green and/or green algae species if the medium is “inoculated” with C Product, i.e., an extract from blue-green algae that has been processed as described above.

It has been determined that one advantage to growing different algae species in a common medium is that the different species of algae will exchange DNA to produce organisms having new properties, and that one of such new properties is producing various new substances that, while not specifically identified on a molecular level, have unique properties.

Once grown together, the mixed algae can be processed as described above.

For the purposes of this disclosure, any algae-based product made according to the disclosed methods and systems that includes a mixture of algae species (e.g., a combination of cyanotic organisms and red algae) is referred to as a “hybrid” product.

Hybrid algae products can include any product made from individual species/types of algae that are grown and processed separately, then mixed. Such hybrid products are more specifically referred to as “post-grown hybrid” products.

Similarly, hybrid algae products can include any product made from multiple species/types of algae that are grown and processed together without the need to mix afterward. Such products are more specifically referred to as “pre-processed hybrid” products.

Because of the above-discussed DNA mixing of different algae species during growth, it is to be appreciated that post-grown hybrid algae products may have appreciably-different properties than pre-processed hybrid algae products.

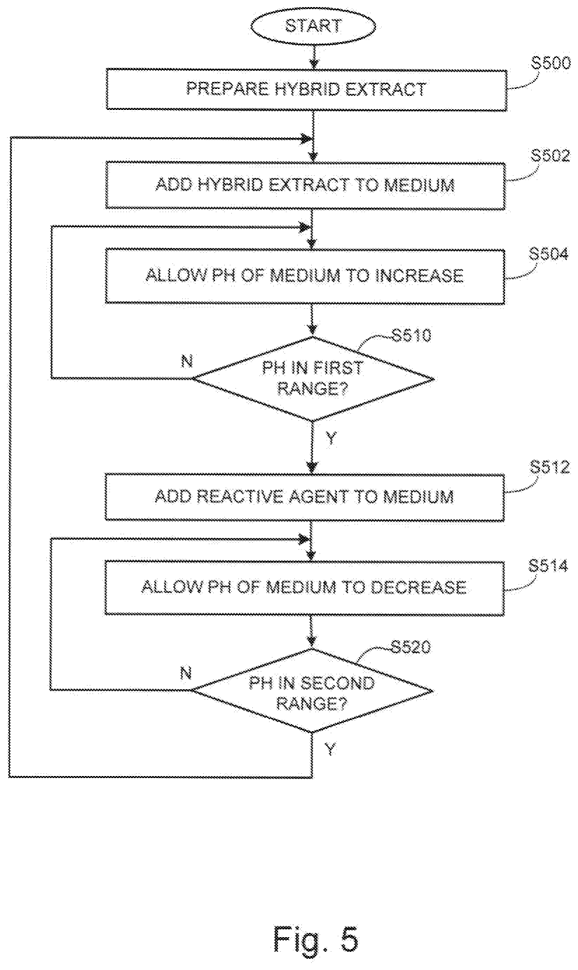

Turning to FIG. 5, a method for performing pollution remediation of a polluted medium is disclosed noting that the term “pollution” may be widely construed. The method starts at S500 where a hybrid extract is prepared. For the purposes of this disclosure, the PH of the hybrid extract is 8.5. However, in various other examples, the PH of the hybrid extract may vary from 9.0 to 8.0, while in other examples the PH of the hybrid extract may vary from 10.0 to 7.5.

In S502, the hybrid extract is added to a medium, which may range from any number of solids or solid mixtures (e.g., soil) and liquids (e.g., fracking water). In various example, the hybrid algae extract may take a dry form produced in the manner discussed above while in other examples it is to be appreciated that it is not essential to reduce the hybrid algae extract to a dry form.

In S504, the PH of the medium is allowed to increase, and in S510, a determination is made as to whether or not the PH of the medium is within a first range. In various examples, the first range may vary from 9.0 to 8.0, and in other examples the first range may vary from 10.0 to 7.0. If the PH of the medium is within the first range, the method of FIG. 5 continues to S512; otherwise, the method jumps back to S504 where S504-S510 repeat until an appropriate PH is reached.

In S512, a reactive agent is added to the medium. In the present example, the reactive agent is a hydrogen peroxide solution dilute to about 20% noting that the example amount of dilution is non-limiting in this disclosure. Further, in other examples the reactive agent may vary among a wide range of acids (e.g., citric acid, sulfuric acid, etc.).

In S514, the PH of the medium is allowed to decrease, and in S510, a determination is made as to whether or not the PH of the medium is within a second range. In various examples, the second range may vary from 6.0 to 5.0, and in other examples the second range may vary from 7.0 to 2.5. If the PH of the medium is within the second range, the method of FIG. 5 jumps back to S502 where more of the hybrid algae extract is added; otherwise, the method jumps back to S514 where S514-S520 repeat until an appropriate PH is reached.

It is to be appreciated that the cycle of operations S502-S520 may be repeated as desired. However, in practice the operations of S502-S520 typically take no more than four cycles.

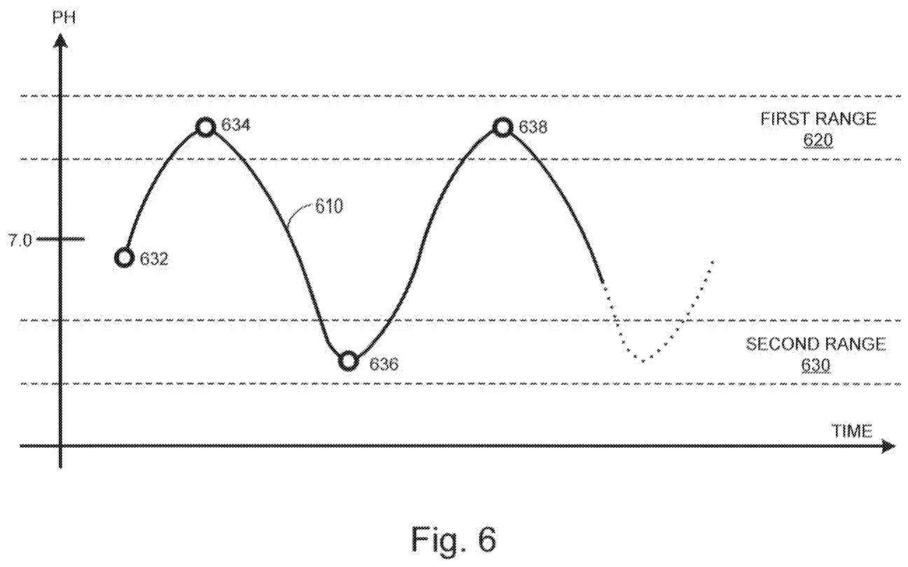

FIG. 6 is a graph of the PH 610 of a medium subjected to the disclosed remediation methods over time. As is shown in FIG. 6 the PH 610 varies to a maximum to some point in the first (upper) range 620 to a minimum to some point in the second (lower) range 630.

Time point 632 represents the PH of the medium when the above-discussed hybrid algae extract is initially added, and time point 636 is another time point (in the second range) where additional hybrid algae extract is added.

Time points 634 and 638 represent points in time when a reactive agent (e.g., hydrogen peroxide) is added.

A particular advantage of the present methods and systems is that the algae-based products increase the efficacy of various reactive agents somewhere between ten to thirty times. For example, a dose of hydrogen peroxide, which at most might last 1-2 minutes, will have its effective time increased to 30-40 minutes using the disclosed methods and systems. The efficacy of other reactive agents will similarly increase. Accordingly, costs related to remediation are substantially reduced.

What has been described above includes examples of one or more examples. It is, of course, not possible to describe every conceivable combination of components or methodologies for purposes of describing the aforementioned examples, but one of ordinary skill in the art may recognize that many further combinations and permutations of various examples are possible. Accordingly, the described examples are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method for pollution remediation, comprising:
growing an amount of cyanotic organisms and red algae;
separating cell walls of the cyanotic organisms from internal portions of the cyanotic organisms to form a separated cyanotic extract, the separated cyanotic extract being the internal portions of the cyanotic organisms;
separating cell walls of the red algae from internal portions of the red algae to form a separated red algae extract, the separated red algae extract being the internal portions of the red algae;
processing the separated cyanotic extract and processing the separated red algae extract so as to create a hybrid algae product; and
performing a pollution remediation operation using the hybrid algae product.

2. The method of claim 1, wherein processing the separated cyanotic extract includes applying an effective amount of alkaline water to the separated cyanotic extract to form a balanced separated cyanotic extract having little or no chemical reactivity, wherein a pH of the alkaline water is a function of a pH of the separated cyanotic extract.

3. The method of claim 2, wherein the cyanotic organisms and the red algae are grown separately, then mixed after forming the separated cyanotic extract and after forming the form the red algae extract.

4. The method of claim 1, wherein:
the cyanotic organisms and the red algae are grown in a common medium; and
forming the separated cyanotic extract and forming the separated red algae extract occur using a common separation operation.

5. The method of claim 1, wherein performing the pollution remediation operation includes:
adding both a reactive agent and the hybrid algae product to a medium containing pollutants and non-pollutants so as to chemically remove the pollutants from the non-pollutants.

6. The method of claim 5, wherein the non-pollutants include at least one of soil and water.

7. The method of claim 6, wherein the reactive agent is hydrogen peroxide, and the sufficient amounts of the hybrid algae product are used so as to at least double the chemically-reactive time of the hydrogen peroxide.

8. The method of claim 7, wherein performing the pollution remediation operation includes:
adding hybrid algae product to the medium containing the pollutants and the non-pollutants so as to increase the PH of the medium; and
subsequently adding hydrogen peroxide to the medium in response to the PH of the medium reaches a first target range so as to decrease the PH of the medium.

9. The method of claim 8, wherein performing the pollution remediation operation further includes:
adding hybrid algae product to the medium in response to the PH of the medium reaching a second target range, wherein the second target range is lower than the first target range; and
again subsequently adding hydrogen peroxide to the medium in response to the PH of the medium reaches the first target range.

10. The method of claim 9, wherein performing the pollution remediation operation further includes:
adding hybrid algae product to the medium and subsequently adding hydrogen peroxide occur at least four times each.

11. The method of claim 9, wherein the hybrid algae product is a post-grown hybrid algae product made from cyanotic organisms and red algae that are grown separately, then mixed together.

12. The method of claim 9, wherein the hybrid algae product is a pre-processed hybrid algae product made from cyanotic organisms and red algae that are grown in a common medium.

13. The method of claim 9, wherein the hybrid algae product is added as a dry-form of coated substrate particles.

14. The method of claim 9, wherein the first target range is from 10.0 to 8.0, and the second target range is from 6.5 to 2.5.

15. The method of claim 14, wherein the first target range is from 9.0 to 8.0, and the second target range is from 6.0 to 5.0.

16. A method of forming an algae-based extract useful for pollution remediation, comprising:
growing an amount of red algae with at least one of cyanotic organisms and green algae in a common medium;
separating cell walls of the red algae and the at least one of cyanotic organisms and green algae to create a purified extract, the purified extract being the internal portions of the red algae and the at least one of cyanotic organisms and green algae;
applying an effective amount of alkaline water to the purified algae extract to form a balanced algae extract having little or no chemical reactivity, wherein a pH of the alkaline water is a function of a pH of the purified algae extract; and
performing a pollution remediation operation using the balanced algae extract.

17. The method of claim 16, further comprising
coating substrate particles with the balanced algae extract to form a dry-form algae product; and
using the dry-form algae product for performing the pollution remediation operation.
